(19) United States
(12) Patent Application Publication
(10) Pub. No.: US 2022/0072171 A1
(43) Pub. Date: Mar. 10, 2022

(54) SHOE SOLE DISINFECTION DEVICE

(71) Applicant: CHENG-CHUNG SUI, Taichung City (TW)

(72) Inventor: CHENG-CHUNG SUI, Taichung City (TW)

(21) Appl. No.: 17/463,387

(22) Filed: Aug. 31, 2021

(30) Foreign Application Priority Data
Sep. 4, 2020 (TW) 109211662

(51) Int. Cl. A61L 2/18; A61L 2/26

(52) U.S. Cl. CPC A61L 2/18; A61L 2202/15; A61L 2202/20; A61L 2/26

(57) ABSTRACT

The invention discloses a shoe sole disinfection device, comprising at least one body, and a receiving structure. The body and the receiving structure are connected to each other by a first main pipe. As a result, the liquid storing in the receiving structure may flow through the first main pipe to the body when the shoes are disposed on the body, and it thus released to the sole for shoes, to enable disinfection for the shoe sole.

### TECHNICAL FIELD

The present invention is related to a disinfection device, specifically indicating to a shoe sole disinfection device.

### BACKGROUND OF RELATED ARTS

The principle of human body cleaning and/or disinfection is to prevent viruses and/or bacteria (such as Escherichia coli, Staphylococcus aureus, Salmonella, etc.) from directly or indirectly invading the human body via attachment to the human epidermis.

In light of the high dependence of the human body on various activities of the hands (such as pressing, picking and other contact behaviors), various investigations, studies, or prevention and treatment of the remaining hands and/or bacteria or viruses have become a popular issue which is needed to pay attention. For example, in public places (such as large exhibition halls, medical institutions, department stores, etc.), there are devices (such as automatic sprayers, pressure sprayers, etc.) for inductive spraying or manual pressure supply of alcohol and/or disinfectant solutions. In addition, according to the average value of the height of the human hand, these disinfection devices are set in public visual field, allowing people's hands to be cleaned and/or disinfected. Furthermore, some people will choose to use portable objects (such as sealed wet wipes, dry-cleaning hand bottles, etc.) containing alcohol or disinfectant solutions to clean or disinfect their hands at any time.

In fact, the use frequency of the feet may as much as the hands. The only difference between the uses of feet and hands is that most people's feet are wrapped in shoes rather than exposed to the environment. However, when the soles of the shoes are contaminated with bacteria or viruses remaining on the ground, unconscious footprints may cause the risk of home hygiene. For example, bacteria or viruses that fall on the floor of the home may indirectly enter the human body through the hands. People are used to pay attention to the maintenance of hand cleanliness, but ignore the pollution which is caused by the feet (shoe soles), leading to the fact that most of the current foot (shoe soles) cleaning-related devices are only installed in the places which require high cleaning conditions such as clean rooms, sterile rooms, etc. It rarely appears in public places (such as department stores, etc.).

On the other hand, in order to automatically transport alcohol and/or disinfectant solution to the surface of the device for shoe sole disinfection via a fixed pipeline which is buried under the ground, the aforementioned foot (or shoe sole) disinfection device is usually fixed directly on the ground or integrally formed on the ground but not according to actual needs. Therefore, when public places or indoor spaces (such as home space or office space) cannot cooperate with constructions such as resurfacing, the device cannot be used for cleaning or disinfecting the feet (shoe soles).

To sum up, with the popularization of health education knowledge, it is expected that people will pay more attention to the cleanliness of their home environment or public places, which will generate related demands and improve the mobility of existing technologies.

### SUMMARY

The purpose of the present invention is to provide a shoe sole disinfection device which is portable or easy to install.

To fulfill the aforementioned purpose, the present invention mainly comprises at least one body and a receiving structure. On the other hand, each of the at least one body comprises a sensing portion, a releasing portion, a plurality of first sub pipes, and at least one second sub pipe. The plurality of first sub pipes are connected to the at least one second sub pipe. The releasing portion includes a plurality of nozzles. The plurality of first sub pipes connected to each nozzle, and the plurality of nozzles are respectively arranged on the recesses of the surface of the at least one body. In addition, the receiving structure is connected to the at least one second sub pipe via a first main pipe. The receiving structure further comprises a chamber, a pumping portion, and a receiving portion. The chamber is connected to the pumping portion, and the receiving portion is electrically connected to the pumping portion. The receiving portion is connected to the sensing portion via signaling. As mentioned above, when the number of the body is plural, the present invention further includes at least one second main pipe for connecting any two of the bodies. The at least one of multiple bodies is connected to the receiving structure via the first main pipe. The various structural features or components of the shoe sole disinfection device provided by the present invention will be described in following implementations. Therefore, the above description is only a brief description of the present invention, and its purpose of it is not to specifically enumerate the key or important elements of the present invention one by one. It is not appropriate to limit the scope of inventions claimed in the present application.

### BRIEF DESCRIPTION OF THE DRAWINGS

FIG. 1 is a schematic diagram of the shoe sole disinfection device of the present invention.

FIG. 2 is a partial structure schematic diagram of the shoe sole disinfection device of the present invention.

### DETAILED DESCRIPTION OF THE INVENTION

The drawings of the present invention will clearly illustrate the embodiments of the present invention as follows. Obviously, the following embodiment is just a part of embodiments of the present invention. It cannot represent all embodiments of the present invention. The disclosure of the following embodiments should be comprised in the scope of the present invention, including the variations which may be thought by a person having ordinary skill in the art.

To realize the technical features of the present application, the embodiment will be specifically described as follows.

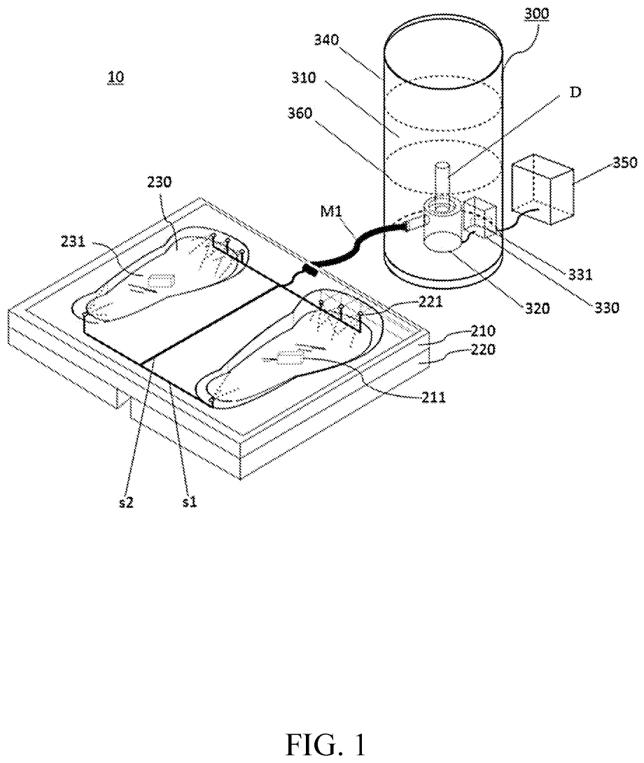

Please refer to FIG. 1, the shoe sole disinfection device 10 mainly comprises a body 200 and a receiving structure 300. The body 200 and the receiving structure 300 are connected by a first main pipe M1. The body 200 has a sensing portion 210, a releasing portion 220, a plurality of first sub pipe s1 and second sub pipe s2. The releasing portion 220 is provided with a plurality of nozzles 221. The receiving structure 300 comprises a chamber 310, a receiving portion 330 and a pumping portion 320. In the present embodiment, the sensing porting 210 is configured under the releasing portion 220. The chamber 310 shares space and connects to pumping portion 320. On the other hand, the receiving portion 330 connects to sensing portion 201 and pumping portion 320 individually via signaling. In this embodiment, the connection between the receiving portion 330 and sensing portions 210 is within a wire (not shown) or wireless, the present invention does not limit thereto. However, in the possible embodiment of the present invention, the sensing portion 201 may be configured in or on the releasing portion 220, the present invention is not limited. The sensing portion 210 is used for detection the situation of releasing portion 220. The aforementioned situation of releasing portion 220 will be used for determining sending a signal or not. The releasing portion 220 connects to the second sub pipe s2 via the first sub pipe s1 and the second sub pipe s2 connects to the first main pipe M1. The chamber 310 is used for storing the pre-released liquid. The signal may determine the receiving portion 330 should drain the pre-released liquid or not from the chamber 310. Of course, the pumping portion 320 is used for executing the draining of the pre-released liquid. When the sensing portion 210 sends a first signal to the receiving structure 300, the releasing portion 220 will spray the pre-released liquid outside the body 200 via nozzles 221. Therefore, the sole will be cleaned and disinfected. The pre-released liquid may indicate to alcohol, disinfectant liquid and other liquids. In other embodiments, the liquid may also be an aqueous solution or a volatile liquid with a cleaning or disinfecting effect.

As shown in FIG. 1, the sensing portion 210 comprises a sensor 211. The sensor 211 may check the existence of the object which is needed to be disinfected. Hence, the receiving portion 330 controls the pumping portion 330 via the signals generated by sensor 211. In other words, the sensor 211 is an infrared sensor and the object which is needed to be disinfected is a shoe sole, but the present invention is not limited thereto. In the other embodiment, the object which is needed to be disinfected may be the naked foot/feet of human. The type of sensor 211 may be ultrasonic sensor, motion sensor or the other type of sensors. The mechanism of the sensor 211 can be set via the intensity of light or temperature. It should be noticed that the sensor 211 is designed to cooperate with the sensing portion 210, therefore to decide the number of the sensor 211 which are needed to be configured on different places such as inside or on the releasing portion 220.

The body 200 further comprises a bearing portion 230. The bearing portion 230 connects to the releasing portion 220. In the practical implementation, the bearing portion 230 is designed as transparent/non-transparent platform which do not disturb the sensing portion 210, avoiding the sensors 211 failing to detect the existence of the object which is needed to be disinfected. For instance, in the embodiment of FIG. 1, a part of the releasing portion 220 forms an inner wall forms the surface of body 200. The bearing portion 230 is configured to attach the extended portion downward the inner wall. On the other hand, the nozzles 221 penetrate the surface of the inner wall and spray the liquid to the bearing portion which is outside the body 200. Furthermore, in order to increase the sensitivity of the sensing area of the bearing portion 230 relating to sensors 211. The bearing portion 230 may be configured to attach and press the upward extended portion of the inner wall. Simultaneously, the structure of the upward extended portion makes the nozzles 221 deform and directly spray the liquid onto the bottom of the bearing portion 230. The sensing quality is increased via the distinction between the sensing of bearing portion 230 and the nozzles 221.

In the other embodiment, the surface of the bearing portion 230 is able to be configured a pre-designed or post-designed decoration 231. The decoration 231 is plane pattern, three-dimensional pattern, concave-convex pattern or combinations thereof. The decoration 231 may be formed by a group of pores. The patterns of the decoration 231 are not specifically limited thereto. In fact, the change of the aforementioned patters should be comprised in the scope of the invention, such as the 2D/3D transformation. Moreover, the decoration 231 can be realized by materials which are the same as the thin plate (bearing portion 230), avoiding to disturb the sensing portion 210. Specifically, if the decoration 231 is a three-dimensional pattern, the three-dimensional pattern can be made by a pre-etching process and then attaches to the bearing portion 230. Therefore, the surface of the bearing portion 230 performs a first area which is formed by convex portions and a second area which is formed by concave portions. The distance, width or the depth of the first area and the second area may be determined by the sensing angle of sensing portion 210, the present invention is not limited thereto.

The nozzles 221 of the present embodiment is a rod-shaped structure which indicates the direction of spraying the pre-released liquid stored in chamber 310 from the body 200 to the object such as shoes. When the opening of the nozzles 221 is oval shape or ellipse shape, the radius or the width per se can be designed as good. In another possible embodiment, the nozzles 221 are atomizing nozzles. The liquid is sprayed as a fog or mist, therefore the bottom of the object such as the sole of a shoe may be covered by a surface layer of micro particles or drips of liquid and the object is disinfected.

In FIG. 1, the receiving portion comprises a receiver 331. The receiver 331 and the sensor 211 are matched by same/similar components. For example, the sensor 211 and receiver 331 may have the matched transmitting circuit, receiving circuit and/or filters. The transmitting circuit, receiving circuit and filters are designed to have similar signaling methods, ways or mechanisms and receive or send the signal with wire or wirelessly. The receiving portion 330 drains the liquid via the signal received by the receiver 331. In an embodiment, when the signal received by the receiver 331 is over a preset threshold, the receiving portion 330 actuates the pumping portion 320 to drain the liquid. However, if the signal received by the receiver 331 is not over a preset threshold, the receiving portion 330 will not actuate the pumping portion 320.

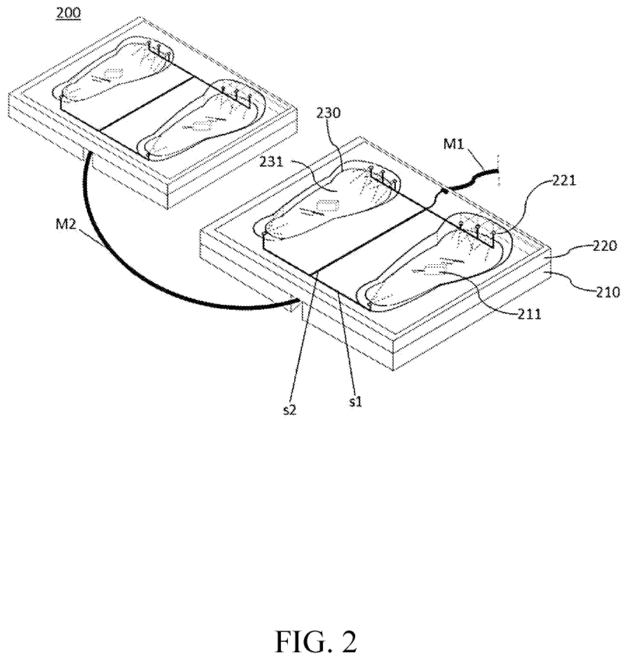

Please refer to FIG. 1 and FIG. 2, FIG. 2 is a partial structure schematic diagram of the shoe sole disinfection device 10 of the present invention. As shown in FIG. 2, each of the nozzles 221 is configured on the outward opening of the first sub pipe s1. The configuration between the nozzle 221 and the first sub pipe s1 is a connection or a unibody form. When the pumping portion 320 is actuated, the liquid will be lead to the nozzle 221 via the first sub pipe s1 and second sub pipe s2 respectively and be sprayed on the bottom of the object such as a shoe sole. When the body 200 is connecting to the receiving structure 300 (as shown in FIG. 1), the draining tube D is configured inside the receiving structure 300 and inward to the chamber 310. A terminal of the draining tube D is immersed into the liquid. On the other hand, the other terminal of the draining tube D is configured inside the pumping portion 320 and thus to connect to the first main pipe M. The liquid can be drained to nozzles 221 via the draining tube D. In the further embodiment, the draining tube D may further comprise a valve which controls certain amount of liquid which is supplied. If the first sub pipe s1 and the second sub pipe s2 are filled with such liquid, the valve will close the terminal of the draining tube D which is immersed into the liquid, stopping the draining of liquid. Furthermore, the valve can be configured in the body of the draining tube D. This configuration may control the motion of liquid to the first main pipe M1 when the first sub pipe s1 and the second sub pipe s2 are full. The configurations of the aforementioned first sub pipe s1 and second sub pipe s2 are formed by a single pipe which matches with multiple pipes, multiple pipes which matches with a single pipe or an unibody structure.

In the embodiment of FIG. 1, the receiving structure 300 further comprises a shell 340 and a power 350. The shell 340 defines the boundary of chamber 310 which is configured in the receiving structure 300. The power 350 is electrically connects to the pumping portion 320 and supplies the power for pumping portion 320. In the present embodiment, the power 350 may be an external power which connects to the pumping portion 320. On the other hand, the power 350 may further connect to (or comprise) a switch. The switch may be manually or remotely controlled therefore to control the ON/OFF of the power 350. Furthermore, a backup power 350 may be connected to the switch and the switch can determine the power supplier of the pumping portion 320.

It is to say that the pumping portion 320 is designed as a pressure pump. In fact, the liquid motion or the velocity per se between the chamber 310 and nozzles 221 can be controlled via adjusting the parameters of the pressure pump such as the pressure. The pumping portion 320 is also able to be air pump, liquid pump or the other pumps which can drain liquid.

As the description mentioned above, the receiving structure 300 further comprises a wall 360. The wall 360 and the shell 340 form the chamber 310, thus to distinct the areas of the chamber 310 and the pumping portion 320. Therefore, as shown in FIG. 1, when the installation of the receiving structure 300 is finished, the draining tube D penetrates the wall 360 and immerses one of the terminals per se into the liquid, for draining the liquid. In the present embodiment, the number of the wall 360 is one, but in the other embodiments, the number of the walls 360 can be two or more, the present invention does not limit thereto.

When the number of the body 200 of the shoe sole disinfection device 10 are multiple, one of the bodies 200 can connects to the receiving structure via the first main pipe M1. On the other hand, the connections between the bodies 200 can be realized by second main pipes M2. The liquid can be supplied into the second sub pipe s2 of each body 200 via the second main pipe M2 and drained to nozzles 221 of every body 200 thus to spray outside the body 200. In some embodiments, the second main pipe M2 having a check valve only allows the unidirectional liquid flow between bodies 200. That is, the reverse flow of the liquid may be stopped between the bodies 200. The second main pipe M2 generally comprises certain types of valve, and the work types of the body 200 and receiving structure 300 are basically the same as the preceding description.

As understood by a person skilled in the art, the foregoing preferred embodiments of the present application are illustrated of the present application rather than limitations of the present application. It is intended to cover various modifications and similar arrangements included within the spirit and scope of the appended claims, the scope of which should be accorded the broadest interpretation to encompass all such modifications and similar structures. While the preferred embodiment of the invention has been illustrated and described above, it will be appreciated that various changes can be made therein without departing from the spirit and scope of the present application.

What is claimed is:

1. A shoe sole disinfection device, comprising:
at least one body, each of the at least one body comprising:
a sensing portion;
a releasing portion, comprising a plurality of nozzles, and the plurality of nozzles are inwardly configured on surface of the at least one body from a part of the releasing portion;
a plurality of first sub pipes, configured on the at least one body and connected with the plurality of nozzles;
at least one second pipe, connected with the plurality of first sub pipes;
a receiving structure, connected with the at least one second pipe via a first main pipe, the receiving structure comprising:
a chamber;
a pumping portion, connected with the chamber; and
a receiving portion, electrically connected with the pumping portion and connected with the sensing portion via signaling.

2. The shoe sole disinfection device as claimed in claim 1, wherein the at least one body further comprising a bearing portion; wherein the bearing portion is connected with the releasing portion and alternatively touches the sensing portion.

3. The shoe sole disinfection device as claimed in claim 1, wherein the receiving structure further comprising:
a shell, defining outside area of the chamber; and
a power, electrically connected with the pumping portion.

4. The shoe sole disinfection device as claimed in claim 1, wherein the plurality of nozzles is atomizing nozzles.

5. The shoe sole disinfection device as claimed in claim 1, wherein the sensing portion comprising a sensor and the sensor is an infrared sensor, an ultrasonic sensor or a laser sensor.

6. The shoe sole disinfection device as claimed in claim 2, wherein the at least one bearing portion is a thin plate.

7. The shoe sole disinfection device as claimed in claim 2, wherein a decoration is configured on an outward surface of the at least one bearing portion; wherein the decoration is plane pattern, three-dimensional pattern, concave-convex pattern or combinations thereof.

8. The shoe sole disinfection device as claimed in claim 3, wherein receiving structure further comprises a wall; wherein the wall and the shell define the chamber which is separated from the pumping portion; and a draining tube, penetrating the wall to the chamber; wherein the chamber and the pumping portion connect to each other.

9. The shoe sole disinfection device as claimed in claim 3, wherein the pumping portion is a pressure pump.

10. The shoe sole disinfection device as claimed in claim 1, wherein the at least one body is multiple bodies, the multiple bodies are connected via at least one second main pipe; wherein one of the multiple bodies connects to the receiving structure.
